(19) United States
(12) Patent Application Publication
(10) Pub. No.: US 2016/0100825 A1
(43) Pub. Date: Apr. 14, 2016

(54) Device to facilitate urine specimen collection

(71) Applicant: MUSC Foundation for Research Development, Charleston, SC (US)

(72) Inventor: Brittan Gotbeter, Charleston, SC (US)

(21) Appl. No.: 14/880,459

(22) Filed: Oct. 12, 2015

Related U.S. Application Data
(60) Provisional application No. 62/062,497, filed on Oct. 10, 2014.

(51) Int. Cl. A61B 10/00

(52) U.S. Cl. CPC A61B 10/007; A61B 2503/06

(57) ABSTRACT

A urine collection system comprised of a plastic liner that releasably mounts to the toilet bowl, rim, or seat with a basin in the center thereof for the collection of urine. The basin has a stopped port to drain the urine into a standard specimen container. The system may be sized to fit a toddler potty or may be sized for a full-sized toilet.

### TECHNICAL FIELD

This invention relates to a device to facilitate collection of a urine specimen.

### BACKGROUND

A urine specimen is routinely ordered to facilitte urinalysis. The clean catch method is often used to avoid contaminating the urine sample with bacteria that are normally present on the skin of the urinary area. To provide the sample, patients are provided a sterile, lidded container and sent to the restroom. After proper hand washing and genital area preparation, patients remove the lid from the container and begin to urinate into the toilet. The container is then manually placed into the urine stream without touching any skin. Afterwards, the lid is placed back on the container and sent for urinalysis. This process is difficult for most to complete hygienically, and is particularly challenging for children, the elderly, hospitalized patients, and anyone in need of assistance.

For example, young potty-trained children are not capable of manually holding the specimen container themselves. This necessitates a parent or other care provider to hold the container while the child urinates. The difficulty is compounded if the child is not comfortable yet on a full-sized toilet, as the parent or care provider must then try to hold the child while simultaneously catching urine in the specimen container.

For all patients the process can be unsanitary. For those patients requiring assistance with holding the specimen container, the process can be unsanitary, embarrassing as well as time consuming since it is common for patients to have difficulty passing urine on demand. If obtaining a urine specimen is not possible using the above process, the ordering physician may result to catheterizing the patient.

Various devices have been contemplated in the art to facilitate obtaining a urine specimen in a more hygienic manner. For example U.S. Patent Application No. 2014/0135651 A1 describes a funnel configured to mate with at least two different urine containers. However, a disadvantage of this and other similar art is that the funnel must still be manually positioned, which is not possible or very difficult for some patients thus still necessitating a care provider to hold the funnel and container.

To avoid the necessity of manually holding the urine specimen container, other collection devices have been described which attach directly to the toilet. U.S. Pat No. 7,011,634 ('634) describes a system in which a collection device shaped as an elongate channel is attached directly to the sample holder. The collection device has attachments that mount on the toilet to position the collection device in the bowl to receive urine. However, this design has several limitations. As the collection device is elongate in shape, it is likely that it will not be properly positioned due to the variable direction of the urine stream. Additionally, the specimen container is threadably connected to the collection device, which limits the number of containers with which the collection device can be used due to difference in threads.

U.S. Pat No. 6,212,698 ('698) describes a urine collection kit for use with a toilet bowl. The '698 patent describes a urine collection kit that mounts to the top of the toilet bowl via a flexible mounting strip. A plastic bag is attached to the mounting strip and depends into the toilet bowl. The plastic bag has an opening reinforced with a collar for urine passage into a specimen container. A specimen container with a support collar is placed into the opening in the plastic bag such that the specimen container support collar is atop the opening collar. A handle attached to the specimen container support collar is used for placement and removal of the container. This setup of this system still requires the patient or caregiver to manually contact urine contaminated surfaces as the support collar and removal handle of the specimen container are both in the path of the urine flow.

U.S. Pat No 5,146,637 ('637) describes a collection system that mounts to the toilet bowl and mates with a specimen container on the underside of the collection system. However, the system requires the specimen container to have a flange at the top edge. Flanged specimen containers are currently not the standard type stocked in healthcare facilities today.

There is a need for a device that facilitates obtaining a urine specimen in a hygienic manner that is compatible with the variety of specimen containers currently used today.

### SUMMARY

As described herein, a urine collection system comprised of a plastic liner that releasably mounts to the toilet bowl, rim, or seat with a basin in the center thereof for the collection of urine. The basin has a stopped port to drain the urine into a standard specimen container. The system may be sized to fit a toddler potty or may be sized for a full-sized toilet.

### DESCRIPTION OF DRAWINGS

FIG. 1 is a urine collection system described in the current invention.

FIG. 2 is a perspective view of the urine collection system inside the bowl of a toddler potty.

FIG. 3 is a representative toddler potty for use with the urine collection system.

FIG. 4 is a representative toddler potty seat for use with the urine collection system.

### DETAILED DESCRIPTION

The present invention now will be described more fully hereinafter with reference to specific embodiments of the invention. The invention can be embodied in many different forms and should not be construed as limited to the embodiments set forth herein.

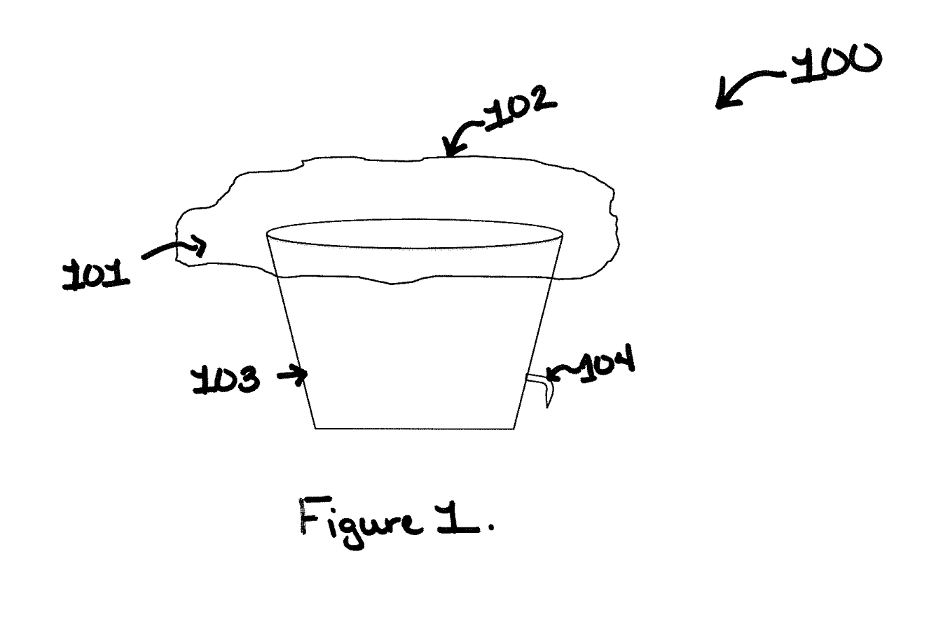

A urine collection system is described to facilitate more hygienic urine specimen collection. The system described can be used with any specimen container. As shown in FIG. 1, the collection system 100 includes a plastic liner 101 coupled to a basin 103. The liner 101 has a retention feature 102 provided along the edge. The basin 103 has a stopped port 104 extending the sidewall near the bottom surface.

The port 104 may be stopped using any number of means known in the art. For example, the stop may be done using a cap, an internal valve, or a stopcock. Port 104 may be made of rigid material or may be pliable such that valve opening is facilitated by pinching the port.

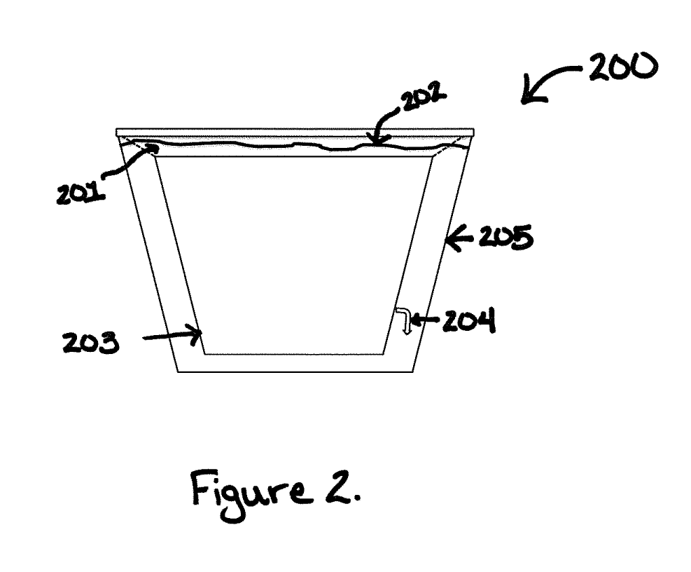

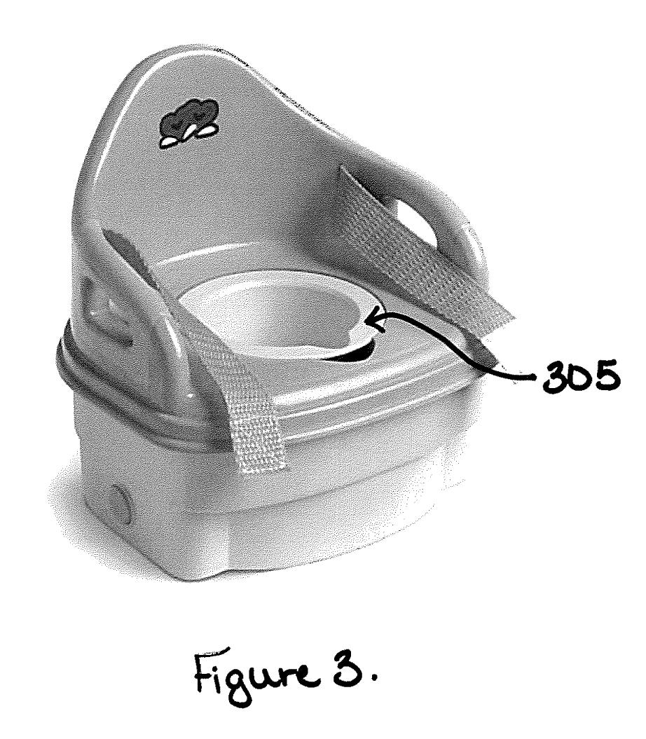

In an embodiment shown in FIGS. 2-3, the collection system 200 is designed for use with a toddler potty 300. As shown in FIG. 2, the collection system 200 depends into the bowl 205 of a toddler potty. Basin 203 is contained within bowl 205. Plastic liner 201 extends from the basin 203 over the edge of bowl 205 and is held in place by retention feature 202, preferably elastic. A non-limiting example of the type of toddler potty that the system can be use with is shown in FIG. 3 in which the collection system 200 shown 100 in FIG. 2 would depend inside the bowl 305 shown in FIG. 3. It should be recognized that bowl 305 comes in several shapes and that plastic liner 201 can be sized to encase all varieties.

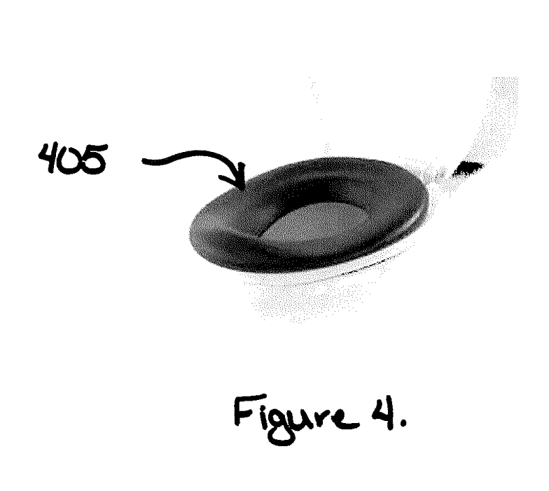

In an alternative embodiment, collection system 100 can be used with a toddler potty seat 405 shown in FIG. 4. When used with potty seat 405, basin 103 of collection 105 system 100 depends into the toilet bowl. Plastic liner 101 encircles potty seat 405 and is held in place by retention feature 102, preferably elastic.

Collection system 100 can be provided in sterile packaging and is meant to be entirely disposable.

### EXAMPLE 1

In use, a patient and/or care provider is provided a specimen container and a packaged collection system 100. After proper hand washing and other instructed preparation, patient or care provider opens the package containing collection system 100. The collection system 100 is removed from the package while taking care not to touch basin 103. Plastic liner 101 of collection system 100 is mounted around potty bowl 205, 305 or potty seat 405, and secured using retention feature 102, such that basin 103 depends into the bowl. The patient then sits down and urinates as normal. Urine is collected in basin 103.

After urinating, the patient or care provider lifts the system 100 from inside the bowl by releasing the retention feature 102. If the retention feature 102 is made of elastic, the patient or care provider simply stretches the elastic to free the collection system 100 from the bowl 205, 305 or seat 405. The collection system 100 can be handled from the edge 102 and outside surface of the plastic liner 101 to avoid manual contact with urine contaminated surface or urine specimen.

Urine collected in basin 103 is then emptied via port 104 into the specimen container. To release the urine through the port 104, the stop is released to allow urine to flow freely from the basin 103.

In alternative embodiments not shown, the collection system 100 is sized for use with a standard toilet. The retention feature 102 may be an adhesive strip, which is adhered to the rim of the bowl under the toilet seat. In this embodiment, a patient or care provider lifts the toilet seat and opens the package containing the collection system 100. The adhesive surface of retention feature 102 is exposed, possibly by peeling away a cover strip. Retention feature 102 is then secured to the rim of the toilet bowl and the seat is lowered. The patient then sits on the toilet seat and urinates normally. Removal and emptying of collection system 100 proceed as described above.

What is claimed:

1. A collection system to facilitate the collection of a urine specimen comprising:
a plastic liner with a retention feature along the outer edge and connected along the inner edge to a basin for the collection of urine;
wherein a stopped port extends from the basin.

2. The retention feature of claim 1, wherein the retention means comprises elastic.

3. The retention feature of claim 1, wherein the retention means comprises an adhesive strip.

4. The stopped port of claim 1, wherein the stopper comprises a cap, a valve or a stopcock.

5. The collection system of claim 1, wherein the system is designed for a toddler potty.

6. The collection system of claim 1, wherein the system is design for a toddler potty seat.

7. The collection system of claim 1, wherein the system is designed for a standard toilet.

8. The collection system of claim 1, wherein the system is provided in a sterile package.
